(19) United States
(12) Patent Application Publication
(10) Pub. No.: US 2002/0156500 A1
(43) Pub. Date: Oct. 24, 2002

(54) Method and device for attaching and /or joining tissue or tissue-like materials to a support

(76) Inventors: Regina Storz-Irion, Emmingen-Liptingen (DE); Reinhard Schnell, Aldingen (DE); Klaus Roth, Ofterdingen (DE); Ludger Schnieder, Kusterdingen-Mahringen (DE); Olivera Josimovic-Alasevic, Berlin (DE); Karl-Gerd Fritsch, Berlin (DE)

Correspondence Address: BRUCE LONDA, NORRIS, MCLAUGHLIN & MARCUS, P.A., 220 EAST 42ND STREET, 30TH FLOOR, NEW YORK, NY, 10017, US

(21) Appl. No.: 10/019,203

(22) PCT Filed: Apr. 20, 2001
(86) PCT No.: PCT/EP01/04544

(51) Int. Cl. A61B 17/04

(52) U.S. Cl. 606/232

(57) ABSTRACT

The invention concerns a method and a device for the securement and/or joining of tissues or tissue-similar materials on a support by laying a suture and by the use of a suturing material.

The task assignment of the invention, to develop a method and a device with which the disadvantages of the state of the art are avoided and with which a reliable, and when required also a liquid-tight and/or a gas-tight securement/joining between tissues of all kinds and a suitable support, particularly also in the form of cartilage, bone hard and soft tissue, is very exactly ensured, is solved in such a way that the tissue to be secured and/or to be joined is placed onto the support, that through the tissue to be secured and/or to be joined and into the support, a first opening is made and into this a suturing material such as a thread is placed, that the thread is fixed-positioned in the opening, that the fixed-positioned thread by means of movement of the device in the suture direction to a further securement and/or joining position is tensioned, and according to the previous steps further openings are made, the thread located in each case, tensioned and fixed-positioned until the required securement/joining and/or the suture length is obtained.

The invention concerns a method and a device for the securement and/or joining of tissues or materials similar to tissues on a support in accordance with the claims 1 and 11.

The securement and/or the joining of tissues or tissue-similar materials on or with a support, respectively, is not only significant in the general form, such as in the furniture industry for example, but also in the special case of medical technology where it is required to join or secure, respectively, biological tissue and/or synthetic tissue with supports such as cartilage, bone, hard or soft tissues.

The securement and/or the joining of tissues on or with supports by means of suturing, nailing, adhesive or fusion methods, respectively, is general state of the art,

In medical technology, and particularly for accident surgery and transplantation medicine, there are instruments and methods available for the securement of tissues which envisage not only the suturing method such as single over-and-over suture or continuous suture, but also the nailing method or the placement of pins.

Where some application areas in medicine are concerned, such as the arthroscopic cartilage cell transplantation, no suturing is possible with instruments and methods according to the state of the art because of the space-restricted conditions in the operating area.

For example, the use of bone pins for the securement of the periost in the knee must be attempted. Performing this particular method of securement requires a high level of skill and involves the inclusion of foreign material as well as requiring sealing with a fibrinous adhesive agent. The time involved for performing the operation is considerable.

In order to achieve a safe performance and a reliable treatment of, for example, defective cartilage, a surgical set of instruments is described in the DE 197 08 703 A1 which uses instruments for cutting, milling, punching, suturing or drilling of bores on cartilage, bone or body tissue or for the injection of operation auxiliary materials.

It is the object of the invention to develop a method and a device with which the disadvantages of the state of the art are avoided and with which a reliable and, when and as required, also a liquid- and/or gas-tight securement/joining between tissues of all types and a suitable support particularly also in the form of cartilage, bone, hard and soft tissue, is ensured to a very exact degree.

According to the invention, the aim of the invention is solved by the features of the claims 1 and 11. Subsequently, the method according to the invention is thus characterised in that the tissue to be secured and/or to be joined is placed onto the support, that through the tissue to be secured and/or to be joined and into the support, a first opening is made and into this a suturing material such as a thread is placed, that the thread is fixed-positioned in the opening, that the fixed-positioned thread by means of movement of the device in the suture direction to a further securement and/or joining position is tensioned, and according to the previous steps further openings are made, the thread in each case is placed, tensioned and fixed-positioned until the required securement/joining and/or the suture length is obtained.

The device according to the invention is thus characterised in that, in an instrument shaft, a tool channel for a tool such as a needle, drill, milling tool, punch, an inlet channel for a suturing thread and for a thread supply and an inlet channel for securing elements and for a supply of securing elements, each having a lower opening, are envisaged, where the lower openings of the channels lead into a common working channel which is formed by the lower section of the tool channel and where, connected with the tool, there is an actuator with which a defined pressure is exerted on the tool and a defined movement of the tool is executed, and where servicing elements for releasing the securing elements, for the thread feed and for the thread tensioning are envisaged.

With the method and the device, it is possible to lay a continuous suture which not only fix-positions the tissues to one another, but also seals off liquid-tight and/or gas-tight against each other.

The suture according to the invention indicates a favourable distribution of forces, so that high durability and elasticity of the joined locations are ensured.

When used in medical technology, shorter operating times are achieved by means of the use of the device compared with the manual method of suturing according to the state of the art, and the use of foreign material for sealing off purposes is avoided. The introduction of foreign materials into the body is reduced to a minimum. It is possible to dispense with all arthrotomy.

Purposeful embodiments of the invention are stated in the Subclaims.

The invention is described as follows in greater detail in an embodiment involving a cartilage defect. The relevant drawings show the following items:

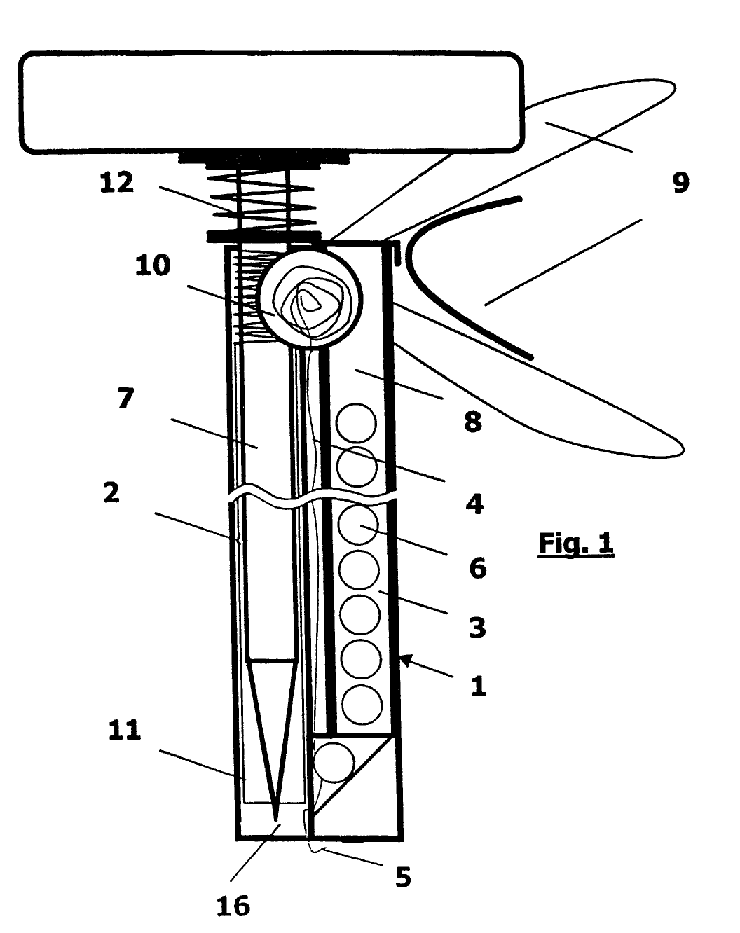

FIG. 1: the schematic arrangement of a first embodiment of the device for the securement of tissues,

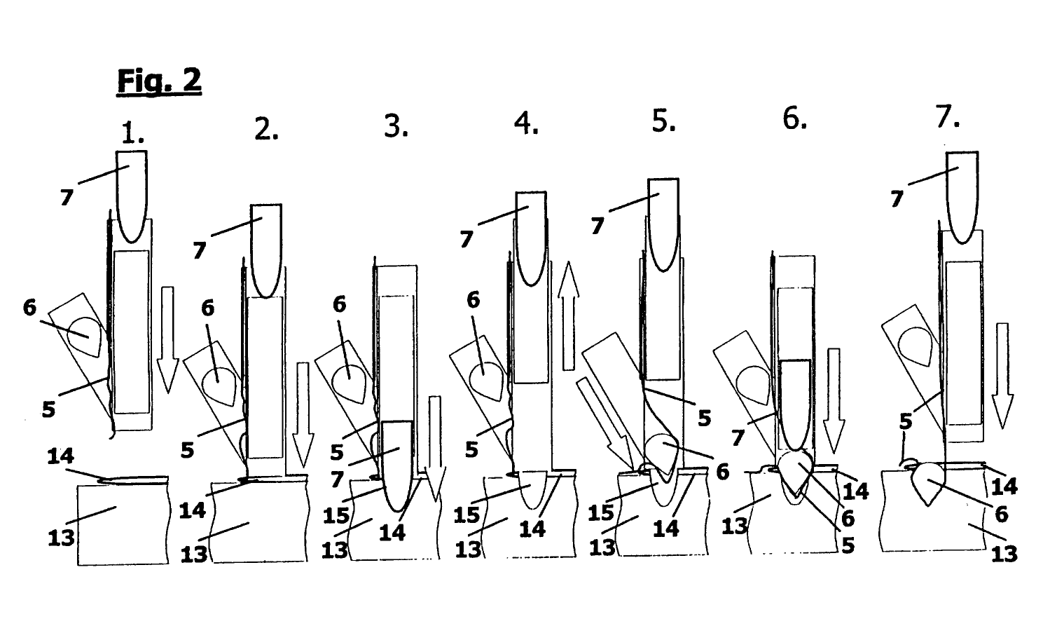

FIG. 2: the schematic arrangement of a sequence of steps according to the method with the use of the device as per FIG. 1,

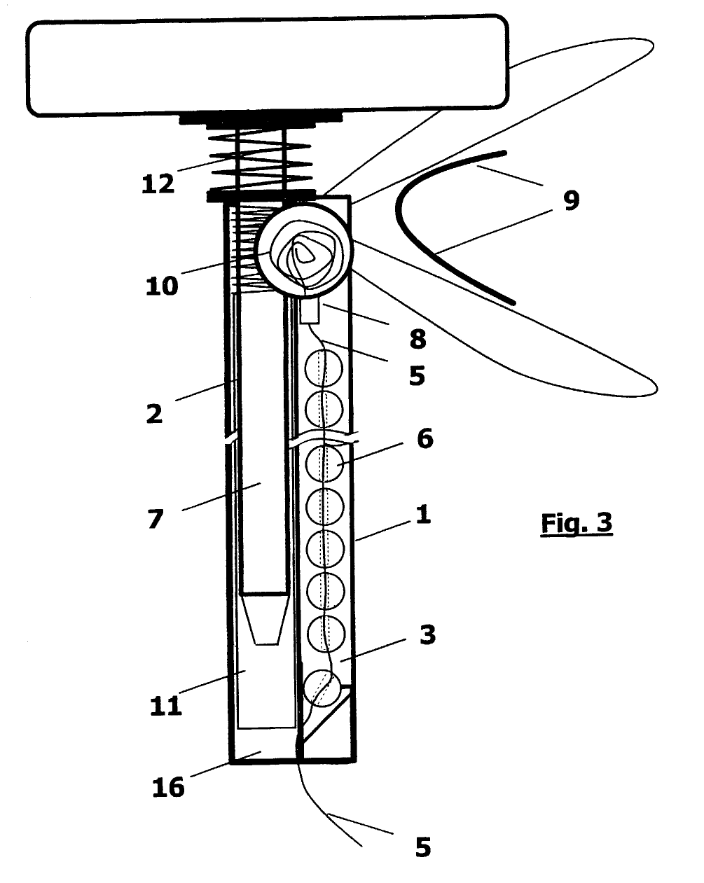

FIG. 3: the schematic arrangement of a second embodiment of the device for the securement of tissues,

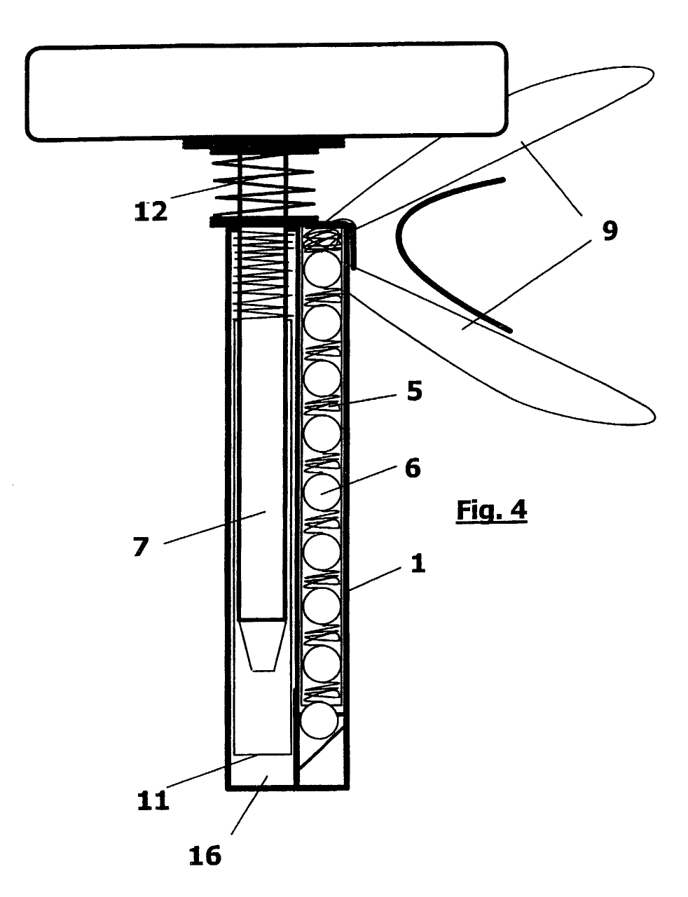

FIG. 4: the schematic arrangement of a third embodiment of the device for the securement of tissues,

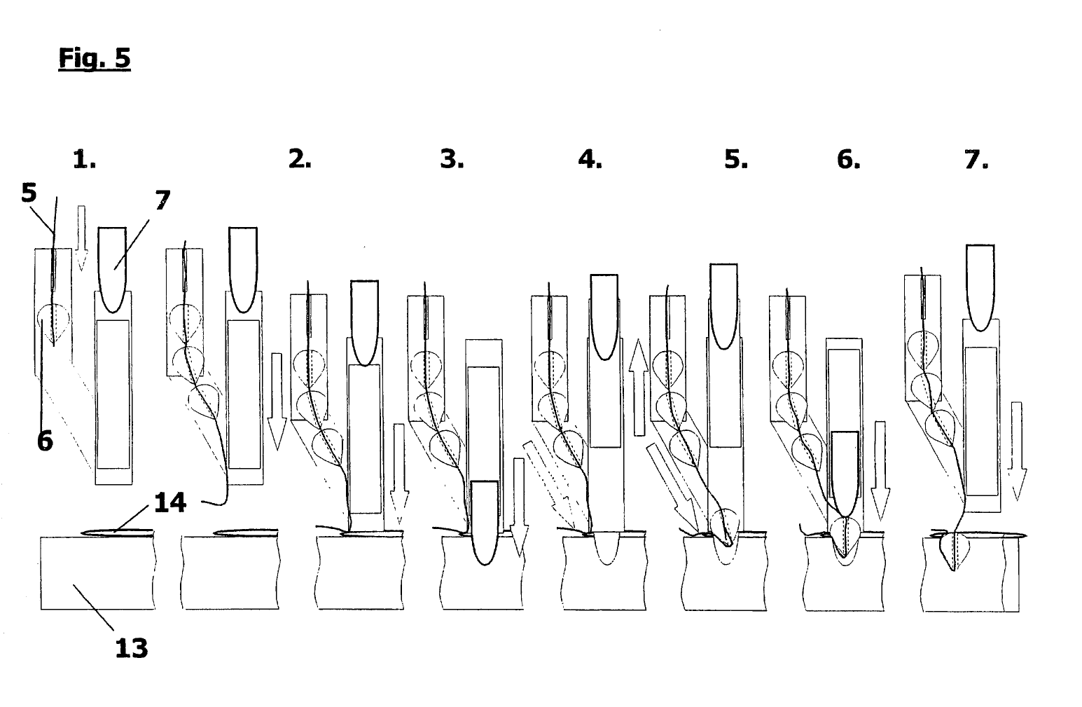

FIG. 5: the schematic arrangement of the sequence of steps according to the method with the use of the device as per FIG. 3, and

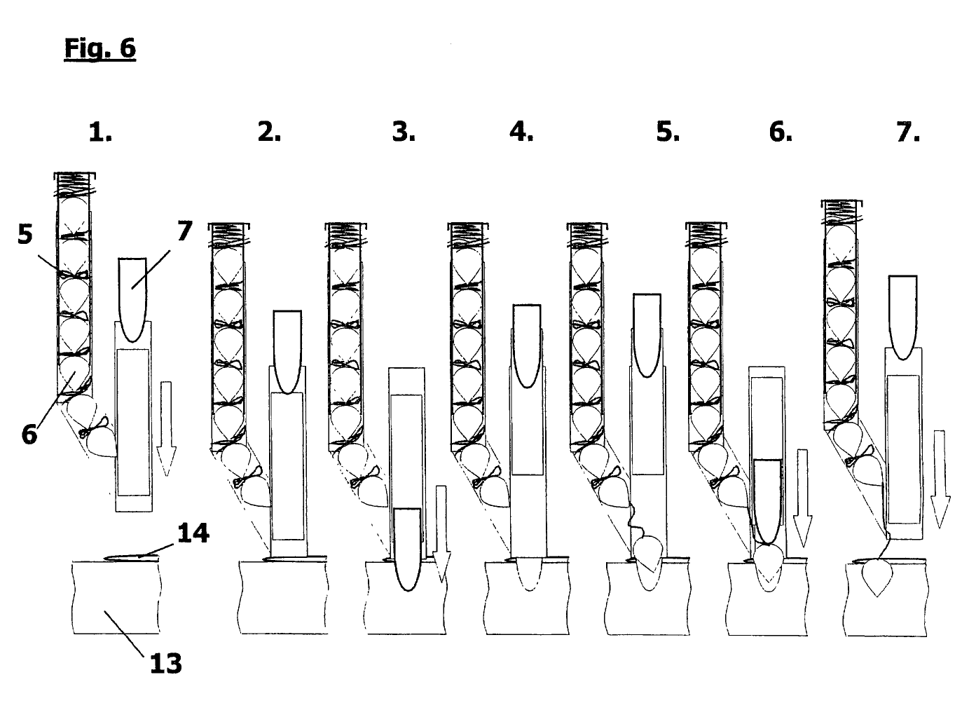

FIG. 6: the schematic arrangement of the sequence of steps according to the method with the use of the device as per FIG. 4.

In accordance with the illustration in FIG. 1, the device according to the invention essentially comprises an instrument shaft 1, into which are located a tool channel 2 for a tool 7 in a sleeve 11, an inlet channel 4 for a suturing thread 5 and for a thread supply 10, an inlet channel 3 for securing elements 6, and with which an actuator 12 and servicing elements 9 are connected.

With this device according to FIG. 1, the steps 1, to 7, are executed for the securement of a tissue 14 onto a cartilage 13 in accordance with FIG. 2.

In a first step 1., the tissue 14 is placed on the cartilage 13. The steps 2 and 3 show that, with the tool 7 and through the tissue 14, a first opening 15 is made into the cartilage 13, and into this opening the thread 5 is placed and, after the tool 7 in step. 4 has been sufficiently drawn back from the opening 15, is fixed-positioned with the steps 5 and 6, through the securing element 6 sliding into the opening 15 The thread 5 in this case runs in front of the opening of the inlet channel 3 (FIG. 1). By means of pressure exertion on the distal side of the inlet channel 3, the securing element 6 is pushed out of the supply stock 8 (FIG. 1) and, at the same time, the thread 5 lying in front of the channel 3 is placed into the opening 15 (FIG. 2) and fixed-positioned. The holding force can be increased by again leading the tool 7 in a downward direction onto the securing element 6 and, subsequently, pressure is exerted on the securing element 6 so that this has a force-locking adaptation to the geometry of the opening 15 or locks in the opening 15. In step 7, the device is led further to the next position and, with this, the entrained thread 5 is tensioned. The steps 2 to 6 are carried out repeatedly until such time as the securement is achieved.

The opening depth to be applied depends on the material of the support and on the geometry of the securing element, and must be selected in such a way that the maximum possible degree of force-locking is achieved. Here, the securing element should not protrude beyond the opening but should close off in a flushing manner.

If the thread 5 is kept under tension while being located, then the finished suture not only serves the fixed-positioning of the tissue, but is also liquid- and gas-tight, respectively.

The inlet channels 3, 4 for the thread 5 and the securing elements 6 and the tool channel 2 can lead into a common working channel 16 (FIG. 1).

The actuator 12 for the individual device components can be executed as a purely mechanical device such as a spring type activation It can also be performed pneumatically, hydraulically or electrically, or also in combinations thereof.

The securing elements 6 can either be in a single arrangement (FIG. 1), loose with the thread 5 (FIG. 3) or solidly connected similar to that of a “string of pearls” (FIG. 4) The sequence of steps with the use of the devices according to the FIGS. 3 and 4 can be seen from the illustrations in the FIGS. 5 and 6.

The devices according to the FIGS. 3 and 4 facilitate thread guidance because the loose or solid connection of the thread 5 with the securing element 6 already provides for a guidance and, in the case of the solid connection between thread 5 and the securing element 6, it allows a portioning of the thread length.

The opening 15 made in the tissue 14 to be secured and in the cartilage 13 can either be drilled, milled, punched, pressed or made in any other way.

The securing element 6 can either be a ball, a bolt, a pin or any other suitable body which fix-positions the thread 5 in the opening 15 in a farce-locking and/or positive locking manner. A fixation of the thread 5 is also conceivable by means of a filling material or an adhesive agent.

The defined movement of the tool 7 can also be of an impulsive nature, for example the shoot-in of a bolt as a securing element 6. Drilling is not required here and can be dispensed with. In such a case the thread 5, connected with the bolt 6, is put into the base and is fixed-positioned by this bolt in the opening 15.

Where the joining in the soft part tissue is concerned, the securing elements 6 can be executed as anchors or as dowels because a force-locking or a positive locking connection is not possible.

The thread 5 and the securing elements 6 can be made from reabsorbing materials.

It is possible to lay a continuous suture which not only fix-positions the tissues to one another, but also seals off against each other when performed in a suitable manner.

If and when required, other materials such as cell dispersions, salt solutions, active ingredients or filling materials or similar substances can be applied to the space between the tissues.

A purse string type suture can also be produced with the device.

For a safe and reliable usage of the device according to the invention, it is purposeful to use the set of surgical instruments according to the DE 197 08 703 A1.

With the set of surgical instruments, the device is positioned into, for example, the knee to be operated and the periost lobe is secured with the first stitch at the preparation boundary in the knee and, when rolling off the applicator, is solidly stitched all around step by step. In this case, the spacing between the stitches is variable. At the same time, the thread is entrained and tensioned before fixation. For this reason, the periost lobe is sealed off at the periphery. A sealing with a fibrinous adhesive agent is not required. The surgical set of instruments ensures that the device always stitches on the outer periphery of the periost lobe.

1. Method for attaching and/or joining tissues or tissue-like materials to a support by means of laying a suture and using a suture material, where
a) the tissue to be secured and/or to be joined is placed on the support,
b) through the tissue to be secured and/or to be joined, and into the support, a first opening is made, and therein the suture material such as a thread is placed
c) the thread is fixed-positioned in the opening,
d) the thread is additionally tensioned by the fixed-positioning process,
e) the fixed-positioned thread by means of movement of the device in the suture direction is tensioned to a further securement and/or joining position and
f) in accordance with the steps b) to d), further openings are applied, the thread is placed in position in each case, fixed-positioned and tensioned, until the required securement and/or joining and suture length, respectively, is achieved.

2. Method according to claim 1, wherein the support is formed from cartilage, bone, hard and/or soft tissue.

3. Method according to claim 1, wherein materials are applied to the space between the tissue to be secured and/or the tissue to be joined and the support.

4. Method according to claim 3, wherein the materials are cell dispersions, salt solutions, active ingredients and/or filling materials.

5. Method according to claim 1, wherein the suture material for the liquid-tight and/or gas-tight joining is placed in location in a pre-tensioned state.

6. Method according to claim 1, wherein the thread is fixed-positioned by at least one securing element such as a ball, bolt or pin and in a force-locking and/or positive locking manner.

7. Method according to claim 6, wherein the securing elements are executed as anchors and/or dowels.

8. Method according to claim 6, wherein the thread and/or the securing elements are made of biocompatible material.

9. Method according to claim 1, wherein the thread is fixed-positioned by a filling material and/or an adhesive agent.

10. Method according to claim 6, wherein the securing elements are connected with the thread.

11. Device for attaching and/or joining tissues or tissue-like materials on a support through a suture and by means of a suture material such as thread, where in an instrument shaft a tool channel (2) for a tool (7) such as a needle, drill, milling cutter, punch are envisaged for making an opening through the tissue to be secured and/or to be joined and into the support, and where an inlet channel (4) for the suture material such as suturing thread (5) and a thread supply stock (10) and an inlet channel (3) for securing elements (6) for the fixation of the suturing thread (5) in the opening and for a supply stock of securing elements (8), each having one lower opening, are envisaged, and where the lower openings of the channels (2,3,4) enter into a common working channel (1) which is formed by the lower section of the tool channel (2), and where with the tool (7) an actuator (12) is connected with which a defined pressure is exerted on the tool (7) and a defined movement of the tool (7) can be executed, and where servicing elements (9) for releasing the securing elements (6), for thread forward-feed and for thread tensioning are envisaged.

12. Method according to claim 11, wherein the actuator (12) is a mechanical, hydraulic, pneumatic, electrical or a combined actuator.

13. Method according to claim 11, wherein the securing elements (6) are formed by solid, formed bodies such as balls, bolts, pins.

14. Method according to claim 11, wherein the securing elements (6) are formed by filling materials and/or elastic bodies and/or adhesive agents.

15. Method according to claim 11, wherein the securing elements (6) are executed as anchors and/or dowels.

16. Method according to one of the claims 11 to 15, wherein the suturing thread (5) and/or the securing elements (6) are made of biocompatible materials.

17. Method according to one of the claims 11 to 16, wherein the securing elements (6) are connected with the thread (5).

18. Method according to one of the claims 11 to 17, wherein the support is formed from cartilage, bone, hard and/or soft tissue.
